(19) United States
(12) Patent Application Publication
(10) Pub. No.: US 2023/0346997 A1
(43) Pub. Date: Nov. 2, 2023

(54) COLD PLASMA VAPOR SANITIZER

(71) Applicant: THE REGENTS OF THE UNIVERSITY OF CALIFORNIA, Oakland, CA (US)

(72) Inventors: Richard E. Wirz, Los Angeles, CA (US); Zhitong Chen, Los Angeles, CA (US)

(73) Assignee: THE REGENTS OF THE UNIVERSITY OF CALIFORNIA, Oakland, CA (US)

(21) Appl. No.: 18/005,954

(22) PCT Filed: Jul. 22, 2021
(86) PCT No.: PCT/US2021/042843

§ 371 Date: Jan. 18, 2023

Related U.S. Application Data
(60) Provisional application No. 63/055,784, filed on Jul. 23, 2020.

(51) Int. Cl. A61L 2/14

(52) U.S. Cl. CPC A61L 2/14; A61L 2202/11; A61L 2202/14; A61L 2202/15

(57) ABSTRACT

A cold plasma hand/object sanitizer to safely and effectively deliver plasma vapor for a variety of applications, including the sanitation of hands and objects. The device uses can be powered directly from any standard plug or can be battery-powered for portable applications. Low-cost and proven electronics can provide all device functions. The feeding gas is ambient air from the local environment but can also include seed gases if desired. The liquid used by the device can be water from any source but alternative liquids and additives can be used as desired. The use of room temperature plasma vapor technology could revolutionize surface and hand/object sanitation and could also be considered for the many promising applications for cold atmospheric plasmas.

### RELATED APPLICATION

This Application claims priority to U.S. Provisional Pat. Application No. 63/055,784 filed on Jul. 23, 2020, which is hereby incorporated by reference. Priority is claimed pursuant to 35 U.S.C. § 119 and any other applicable statute.

### TECHNICAL FIELD

The technical field generally relates to a sanitizer device that uses cold plasma. The device operates using only water and air and may be powered by conventional power sources. The device is suitable for use in the home, office, medical facilities (e.g., hospitals or physician’s offices), hotels, building entrances, commercial establishments, and the like.

### BACKGROUND

With the emergence of severe acute respiratory syndrome coronavirus (COVID-19), a newly discovered infectious disease, the importance of primary infection control measures has been highlighted. The need to clean hands frequently with alcohol-based hand rub or soap and water has been cited by the World Health Organization (WHO) as being one of the most important hygiene measures in preventing the spread of infection. This statement has been reiterated by many countries including the Centers for Disease Control (CDC), in reference to reducing the transmission of coronavirus, the influenza virus, and other infectious pathogens. Hands are one of the most frequent transmission routes for many infections where they come in direct contact with known portals of entry for pathogens, such as mouth, nose, and eyes. Hand sanitizer is known and is used to prevent the transmission of infection, which is majorly caused through hand transmission, further causing several diseases such as nosocomial food-borne illness and others. Unfortunately, as the current pandemic involving COVID-19 has shown, conventional hand sanitizers have experienced well documented shortages. Conventional hand sanitizers typically use high proof alcohol such as isopropanol or ethanol as the biocidal agent. Hand sanitizers may be in gel form or spray form. Again, as noted above, there have been well known shortages of hand sanitizers, and existing stocks of isopropanol, for example, have been exhausted. Distilleries, for example, have adjusted operations to formulate high proof ethanol which can be used as a hand sanitizer. There are several downsides, however, to alcohol-based hand sanitizers. First, to be effective, the sanitizer material needs high proof concentrations of at least 60% to have biocidal activity. This high proof requirement, however, has safety implications because at such high proof levels the hand sanitizer is flammable. In addition, the hand sanitizer may be ingested causing intoxication. Moreover, alcohol-based sanitizers often induce skin drying and irritation. There thus is a need for alternative hand sanitizers.

### SUMMARY

In one particular embodiment, a plasma vapor dispensing device and method is disclosed that relies upon so-called cold plasma as a biocidal agent for use as a sanitizer. Recent progress in atmospheric plasma has led to the creation of “cold” plasmas with ions/heavy particles that are close to room temperature, which has tremendous applications in biomedical engineering. While the plasma is referred to as “cold” this does not imply that the plasma is chilled or cooled. In fact, the cold plasma exists at near room temperature. The efficacy of cold plasma can be attributed to the components of the plasma: electrons, charged particles, reactive oxygen species (ROS), reactive nitrogen species (RNS), free radicals, ultraviolet (UV) photons, molecules, electromagnetic fields, physical forces, and electric fields.

The plasma vapor dispensing device leverages these benefits of cold plasma into a dispenser that generates cold plasma using, one embodiment, water and air. The cold plasma vapor that is created is a vapor-based plasma in one embodiment that is mixed with or otherwise contains atmospheric air and then is directed out of the dispensing device onto the intended surface (e.g., hands of the user or an object). The dispenser device includes a number of subcomponents or subsystems that cooperatively operate together to generate the cold plasma from water and air. This includes a reservoir that contains or holds a volume of fluid or water therein. The reservoir, in some embodiments, may periodically be refilled as needed (e.g., from the tap, bottle, or purified source). Of course, the reservoir may also be coupled to a water source through appropriate piping or conduits and valves so that the manual filing of the dispenser is not needed. One advantage of the present invention is the immediate output or ejection of the plasma-treated vapor to the object and/or subject of sanitation. In this regard, the device ensures that a high concentration of plasma species is available to the subject/object. Of course, the device may also be designed to allow for additional space between the plasma-vapor source for distribution of the plasma-treated vapor or to achieve a desired concentration of plasma species.

The dispenser device further includes an aerosolizer or vaporizer. The aerosolizer or vaporizer creates vapor or small aerosolized droplets of fluid (e.g., water). The aerosolizer or vaporizer may, in one embodiment, generate the water vapor via a vibrating substrate or plate (e.g., piezoelectric substrate). The substrate or plate vibrates in response to an applied electrical current. In another embodiment, the aerosolizer or vaporizer may incorporate an injected gas through or over the liquid such that the gas is infused with vapor. The dispenser device further includes an air intake unit such as one or more fans or pumps that pulls and/or pushes in ambient air from the environment of the dispenser device. In other embodiments, at least a portion of the gas may also come entirely or partially from a tank which may be pressurized or unpressurized with one or more seed gases (e.g., Nitrogen gas). One or more plasma generators are located in the dispenser device is a plasma generating region and are in contact with air entering the dispenser device and/or the generated water vapor. In one particular embodiment, plasma is created within the air pulled into the dispenser device using one or more electrodes coupled to an electrical power source. This plasma is then mixed with the generated water vapor in a mixing chamber or the like. The now-plasma-infused water vapor contains reactive oxygen species (ROS) and reactive nitrogen species (RNS). The plasma infused water vapor is then dispensed from the device. One or more fans or gas pumps may optionally aid in dispensing the plasma infused water vapor via one or more outlets or exhausts of the dispenser. In other embodiments, the work of the air intake unit is enough to exhaust or output the plasma vapor. In one embodiment, the dispensed plasma infused water vapor is directed onto the surface of the skin. For example, a user may direct his or her hands underneath the exit vapor flow where the user rubs the hands together covering all surfaces of the hands and fingers. Of course, the plasma infused vapor may be applied to other surfaces as well including surfaces of devices (e.g., medical devices), instruments, utensils, or any surface that requires sanitation for any application (including medicine, food, agriculture, public/private spaces, personal protective equipment (PPE)), and the like.

In one embodiment, a plasma vapor dispensing device includes a fluid reservoir configured hold liquid fluid therein and an aerosolizer/vaporizer configured to generate a vapor of the liquid contained in the fluid reservoir. The plasma vapor dispensing device further includes an air intake unit that pumps or transports ambient air into the plasma vapor dispensing device from the surrounding environment. One or more plasma generating electrodes are operatively coupled to a high voltage power source and disposed in the plasma device at a plasma generating region to generate plasma in the presence of ambient air, the liquid vapor, or a mixture thereof to generate a plasma vapor. One or more outlets that exhaust or output the plasma vapor from the plasma vapor dispensing device.

In another embodiment, a plasma vapor dispensing device includes a fluid reservoir configured hold liquid fluid therein. A source of gas is provided along with an aerosolizer/vaporizer configured to generate a vapor of the liquid contained in the fluid reservoir from gas from the source of gas traveling through or over the liquid fluid. The plasma vapor dispensing device further includes one or more plasma generating electrodes operatively coupled to a high voltage power source and disposed in the plasma device at a plasma generating region to generate plasma in the gas, the liquid vapor, or a mixture thereof to generate a plasma vapor. One or more outlets are present in the plasma vapor dispensing device that exhaust or output the plasma vapor from the plasma vapor dispensing device.

In another embodiment, a plasma vapor dispensing device includes a fluid reservoir configured hold liquid fluid therein and an aerosolizer/vaporizer configured to generate a vapor of the liquid contained in the fluid reservoir. The plasma vapor dispensing device includes an air intake unit that pumps or transports ambient air into the plasma vapor dispensing device from the surrounding environment. One or more plasma generating electrodes are operatively coupled to a high voltage power source and disposed in the plasma device at a plasma generating region to generate plasma in the presence of ambient air, the liquid vapor, or a mixture thereof to generate a plasma vapor. The plasma vapor dispensing device includes one or more outlets that exhaust or output the plasma vapor from the plasma vapor dispensing device. The one or more plasma generating electrodes generate plasma at the plasma generating region in the presence of ambient air and plasma which is directed onto or into the fluid contained in the fluid reservoir. This “activated” fluid can then be used directly to create the plasma vapor which is dispensed from the device.

In another embodiment, a method of using plasma vapor to disinfect or sterilize a surface using a plasma vapor dispensing device includes the operations of: generating water vapor using a vibrating substrate or surface in the plasma vapor dispensing device; pulling ambient air into the plasma vapor dispensing device with an air intake unit; generating a plasma vapor in the presence of ambient air, the liquid vapor, or a mixture thereof with one or more plasma generating electrodes operatively coupled to a high voltage power source and disposed in the plasma device; and exhausting or outputting the plasma vapor from the plasma vapor dispensing device via one or more outlets onto the surface.

In another embodiment, a method of using plasma vapor to disinfect or sterilize a surface using a plasma vapor dispensing device includes the operations of: generating liquid vapor using one or more of: (i) a vibrating substrate or surface in the plasma vapor dispensing device with ambient air and/or (ii) a source of gas that is delivered into or over liquid contained in the plasma vapor dispensing device; generating a plasma vapor in the presence of one or more of the ambient air, the source of gas, the liquid vapor, or a mixture thereof with one or more plasma generating electrodes operatively coupled to a high voltage power source and disposed in the plasma device; and exhausting or outputting the plasma vapor from the plasma vapor dispensing device via one or more outlets onto the surface.

In another embodiment, a method of using plasma vapor to disinfect or sterilize a surface using a plasma vapor dispensing device includes the operations of: pulling ambient air into the plasma vapor dispensing device with an air intake unit; generating a plasma in the presence of ambient air with one or more plasma generating electrodes operatively coupled to a high voltage power source and disposed in the plasma device and contacting the generated plasma with water contained in a fluid reservoir; generating plasma vapor using a vibrating substrate or surface in the plasma vapor dispensing device and in contact with the water in the fluid reservoir; and exhausting or outputting the plasma vapor from the plasma vapor dispensing device via one or more outlets onto the surface. Here the water is activated in response to contact with the generated plasma. The activated water can then be formed into a plasma vapor which is exhausted or output into the desired surface(s).

### BRIEF DESCRIPTION OF THE DRAWINGS

FIG. 1A illustrates a perspective view of a plasma vapor dispensing device according to one embodiment.

FIG. 1B illustrates a schematic view of a plasma vapor dispensing device according to one embodiment.

FIG. 1C illustrates a schematic view of a plasma vapor dispensing device according to another embodiment. This embodiment uses a seed gas contained in a tank.

FIG. 1D illustrates a schematic view of a plasma vapor dispensing device according to another embodiment. This embodiment creates activated fluid (e.g., water) using cold plasma that is contacted with the fluid.

FIG. 2A shows a photograph of a four-electrode configuration (with electrodes configured in a cylindrical channel) that is used to generate plasma vapor (water vapor passing through plasma).

FIG. 2B shows a photograph of cold plasma discharge in air and water vapor at atmospheric pressure.

FIG. 3 indicates optical emission spectroscopy results of reactive oxygen and nitrogen species (RONS) generated by the plasma. A fiber-coupled optical spectrometer (LR1-ASEQ Instruments), with a range of wavelength 300-1000 nm, was employed to detect plasma generated RONS (such as nitric oxide [NO], nitrogen cation [N2+], atomic oxygen [O], and hydroxyl radicals [•OH]).

FIG. 4 illustrates one embodiment of four electrodes in a cylindrical channel (two pairs of electrodes) used to generate plasma vapor. The device may be configured and used in any orientation.

FIGS. 5A and 5B illustrate schematically one mode of separately forming vapor and plasma which are then combined and mixed to form plasma vapor. This, for example, is the mode of operation of FIG. 1B.

FIGS. 6A and 6B illustrate schematically another mode of forming vapor which is passed to the plasma generating region to create the plasma vapor. Plasma is thus formed in the presence of vapor. Mixing may take place along with plasma formation to create a well-mixed -plasma vapor.

FIGS. 7A and 7B illustrate schematically another mode of forming plasma vapor. In this mode of operation, features of the embodiments of FIGS. 5A, 5B, 6A, and 6B are combined. A fraction of the vapor is passed or injected into the plasma generating region, while the rest of the vapor mixes with the plasma vapor in a mixing region to produce a plasma vapor of the desired concentration or with the desired humidity.

FIG. 8 schematically illustrates various modes of forming plasma vapor. This illustrates that at least some of the plasma may be combined with vapor prior to mixing. The plasma may be emitted along with the vapor and subsequently mixed as illustrated. Combinations of the same are also envisioned.

### DETAILED DESCRIPTION OF ILLUSTRATED EMBODIMENTS

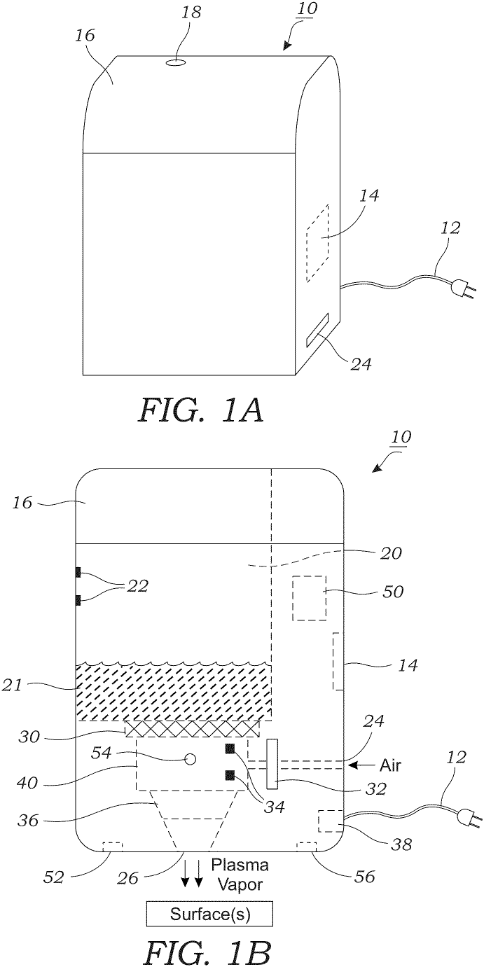

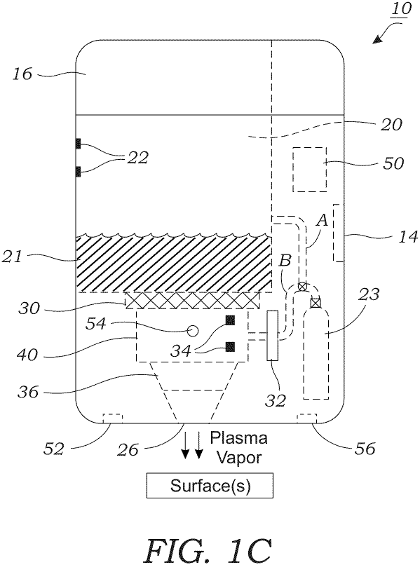

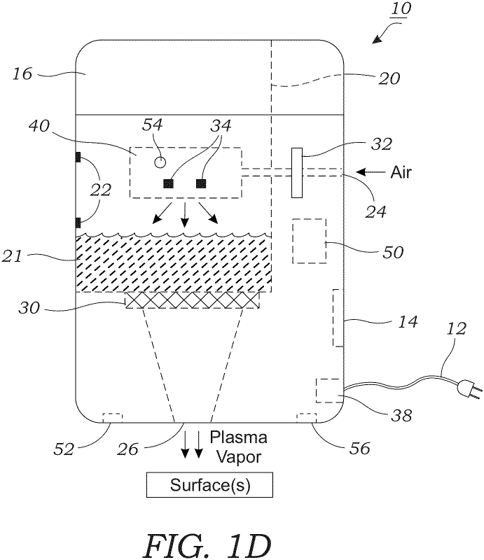

FIGS. 1A and 1B illustrate one exemplary embodiment of a plasma vapor dispensing device 10 according to one embodiment. The plasma vapor dispensing device 10 may, in one preferred embodiment, be used as cold plasma vapor hand/object sanitizer. The term plasma vapor is meant to indicate that the plasma and reactive species therein is/are present in conjunction with a vapor from a fluid such as water. It has been found that the efficacy of the cold plasma is enhanced or improved due to the presence of the vapor in combination with the plasma. Without having to be bound by a specific theory of operation, it is believed that the presence of the vapor (e.g., water vapor) is able to maintain the reactive species of plasma for longer periods of time (as opposed to just air). In addition, the presence of the vapor allows the plasma vapor to contact the surface to be sanitized to create a wetting effect thereon. This wetted surface can then rubbed onto the hands or object much like the way conventional hand sanitizer is used on the hands where is rubbed/worked over the surface of the hands.

The size of the plasma vapor dispensing device 10 may vary but it may be located or situated in various locations. For example, the plasma vapor dispensing device 10 may be the size of a small home appliance (e.g., toaster, microwave, and the like) and may be located on a wall, a stand, or post, or even a countertop. The plasma vapor dispensing device 10 is a powered device that requires electricity to operate. In one embodiment, the plasma vapor dispensing device 10 is powered using a power cord or cable 12 that plugs into a conventional alternating current electrical socket found in residential and commercial buildings. Alternatively (or in addition to), the plasma vapor dispensing device 10 may be battery powered through an internal battery 14 (FIGS. 1A-1D) which may be replaceable or rechargeable. The plasma vapor dispensing device 10 includes an external housing or casing 16 that contains the various components. The external housing or casing 16 may include an inlet 18 (FIG. 1A) that is used to fill a reservoir 20 with a fluid 21. The fluid 21 in one preferred embodiment is water but may be other liquids as explained herein. The inlet 18 may include a connector (e.g., nipple) or the like that can connect to tubing or other conduit that is connected to the source of water or other liquid. The water or other liquid can be from the tap, purified or distilled water, or other sources. Additives may optionally be added to the water or other liquid in certain embodiments. This includes supplements, medicaments, moisturizers, fragrances, and the like. The reservoir 20 may include one or more optional level sensors 22 that may alert the user to when the volume of liquid in the reservoir 20 runs low or needs to be filled. The water may be purified or distilled water. Alternatively, the water may be normal tap water although this may require periodic cleaning to remove mineral scales that develop during the vaporization process. Again, while water is the preferred liquid according to one embodiment, it should be appreciated that other liquids may be used.

In addition, the external housing or casing 16 may include one or more air inlets 24 used which are used to draw ambient air into the plasma vapor dispensing device 10 to generate the cold plasma vapor as described herein. These may be ports, holes, apertures, or louvers formed in the housing or casing 16 which allow air to enter the plasma vapor dispensing device 10. Likewise, the external housing or casing 16 may include one or more outlets 26 that is/are used to direct the cold plasma water vapor to the desired surface(s). The one or more outlets 26 may optionally include directed outlets like nozzles or the like (or directable nozzles). Of course, the output of the cold plasma vapor may occur in any number of configurations. For instance, the cold plasma vapor may be outputted/ejected as one or more streams or even a curtain of vapor.

The plasma vapor dispensing device 10 includes an aerosolizer/vaporizer 30 that generates vapor or droplets from the fluid 21 stored in the reservoir 20. As explained herein, in one preferred embodiment, the fluid 21 is water and the vapor is water vapor. The aerosolizer/vaporizer 30 may include a substrate or plate that vibrates at high frequency in the presence of fluid 21 to generate droplets to create the vapor (e.g., water vapor). For example, a piezoelectric plate or substrate is applied with alternating current and undergoes vibration to produce ultrasonic waves. The ultrasonic waves cause water droplets to form above the surface of the water 21 in the reservoir 20. The aerosolizer/vaporizer 30 is preferably actuated during the actual use of the plasma vapor dispensing device 10 and acts as an on-demand source of water vapor. The plasma vapor dispensing device 10 also includes an air intake unit 32 which is used to draw ambient air into the plasma vapor dispensing device 10 from the external environment via air inlet(s) 24 and, in some embodiments, push the formed plasma vapor out of the plasma vapor dispensing device 10. The air intake unit 32 may include one or more fans or air pumps that pull/push air from the surrounding environment and provide a pressure differential to effectuate gaseous flow through the plasma vapor dispensing device 10. The air intake unit 32 may also pull or push vapor generated by aerosolizer/vaporizer 30 into the mixing region 36 and/or the plasma generating region 40 as explained herein. Alternatively, and with reference to FIG. 1C, in another embodiment, the air or other gas (e.g., an inert gas such as nitrogen, helium, neon, argon) may be injected into or over the liquid (e.g., water) to generate a gas that is infused with or contains vapor from the fluid 21. The air or other seed gas in this embodiment may be stored in a pressurized or unpressurized tank 23 (e.g., bottle or the like) that is injected through the fluid 21 (e.g., bubbled) or passes over the surface of the fluid 21 to create the liquid infused vapor (seen in path A in FIG. 1C). The air or other gas from the tank 23 may also be delivered to the electrodes 34 for generation of plasma which can then be combined with vapor generated by the aerosolizer/vaporizer 30 (seen in path B of FIG. 1C). In some cases, an air intake unit 32 may be needed to assist in delivering air or other gases to the plasma generating region 34 (e.g., if the gas is stored at low pressures, for example). The air intake unit 32 may also pull in ambient air to mix with the air/gas from the tank 23.

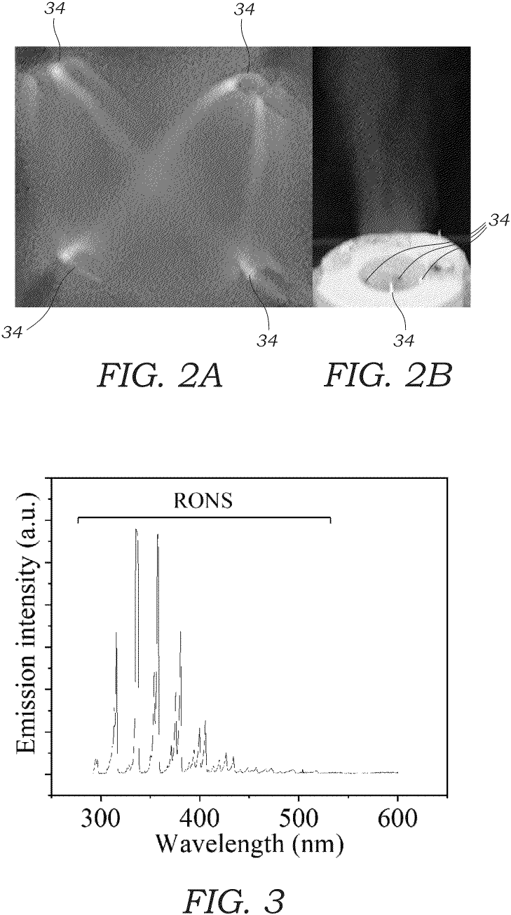

With reference to FIGS. 1A-1D, the plasma vapor dispensing device 10 includes one or more electrodes 34 that are used to generate plasma. The plasma generating electrode(s) 34 includes at least a positive (+) and a negative (-) terminal. Electrical discharge between the +/terminals of the electrode 34 generates the plasma. The electrodes 34 may include any number of shapes or configurations of electrodes including cylindrical, conical, square, rectangular, and the like. The electrodes 34 may be driven using an applied high voltage in one embodiment from high voltage power source 38. In some embodiments, multiple such plasma generating electrode(s) 34 are used to generate the plasma. FIGS. 2A and 2B, for example, illustrates two pairs of electrodes 34 (i.e., four electrode configuration). Using larger numbers of electrodes 34 (or larger electrodes 34) may be used to more efficiently generate plasma-exposed water vapor with a high concentration of ROS and RNS.

FIG. 3 indicates optical emission spectroscopy results of reactive oxygen and nitrogen species (RON) generated by the plasma from an electrode configuration illustrated in FIGS. 2A and 2B. A fiber-coupled optical spectrometer (LR1-ASEQ Instruments), with a range of wavelength 300-1000 nm, was employed to detect plasma generated ROS and RNS (such as nitric oxide [NO], nitrogen cation [N2+], atomic oxygen [O], and hydroxyl radicals [•OH]).

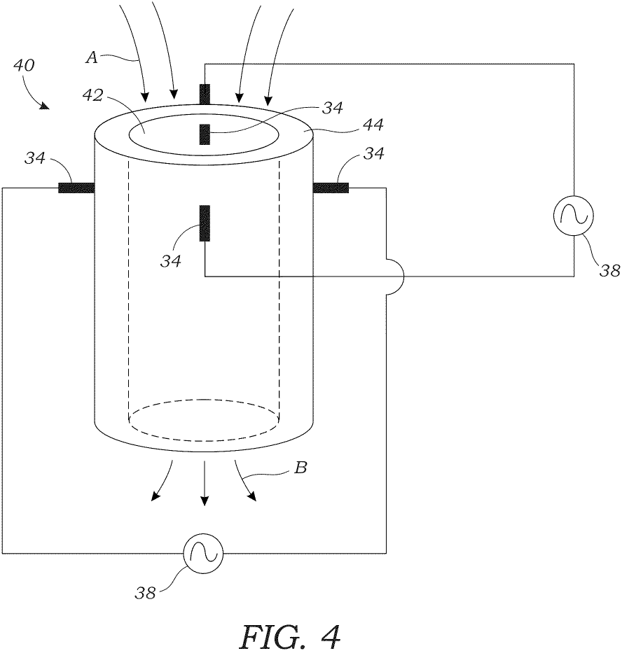

FIG. 4, for example, illustrates one embodiment of a plasma generating region 40 located in the plasma vapor dispensing device 10 that uses four (4) cylindrical electrodes (each 0.6 mm diameter and 5 mm in length) used to generate plasma vapor. The plasma generating region 40 is formed in a flow path 42 that is formed in a collar or nozzle 44 that has the electrodes 34 disposed thereon/therein. Plasma is generated between the two opposing electrodes 34 that form a pair when connected to a high voltage power source 38. Arrows A in FIG. 4 indicate the flow of air, air/gas, or fluid vapor entering the plasma generating region 40. Arrows B in FIG. 4 indicate the flow of the formed plasma vapor exiting the plasma generating region 40. As seen in FIG. 4, there could be multiple high voltage power sources 38 as illustrated with one power source 38 for a pair of electrodes 34. Alternatively, a single high voltage power source 38 may be used to power the electrodes simultaneously or alternatingly using, for example, a switch or the like to switch different pairs of electrodes 34. The peak-to-peak voltage generated by the high voltage power source 38 may vary but is typically within the kilovolt range and more typically within a range between about 1-10 kV. An exemplary peak-to-peak voltage may include 8 kV. The high voltage power source 38 is, in one preferred embodiment, a high voltage alternating current (AC) power source. The high voltage may be generated using a step-up transformer/converter. In some embodiments, where the device is powered by a battery, the pair of electrodes 34 are powered by DC-to-AC converters with integrated step-up transformers/converters (for each electrode pair). In some embodiments, the device 10 may utilize pulsed DC voltages between the electrodes 34 to generate the plasma. High voltage DC-to-DC step-up converters may be used in some embodiments. The plasma vapor dispensing device 10 is easy to recharge/plug-in due to it being compatible with different types of source plugs, voltages (e.g., 110-250 V), and frequencies (e.g., 50-60 Hz) around the world.

In some embodiments, the frequency and/or the peak-to-peak voltages used to power the electrodes 34 may be adjusted. For example, different applications may use different frequencies and/or the peak-to-peak voltages. Use of the plasma vapor dispensing device 10 on living tissue may be different than operating conditions used for non-living objects. In addition, different pathogens may demand different frequency and/or the peak-to-peak voltages. These may be adjusted by the user using a dial, button, or the like. Alternatively, the plasma vapor dispensing device 10 may be programmed with different operating settings that may be selected by the user (e.g., use for hands, use for pathogen X, etc.). These may be selected by a dial, button, or user interface in the plasma vapor dispensing device 10.

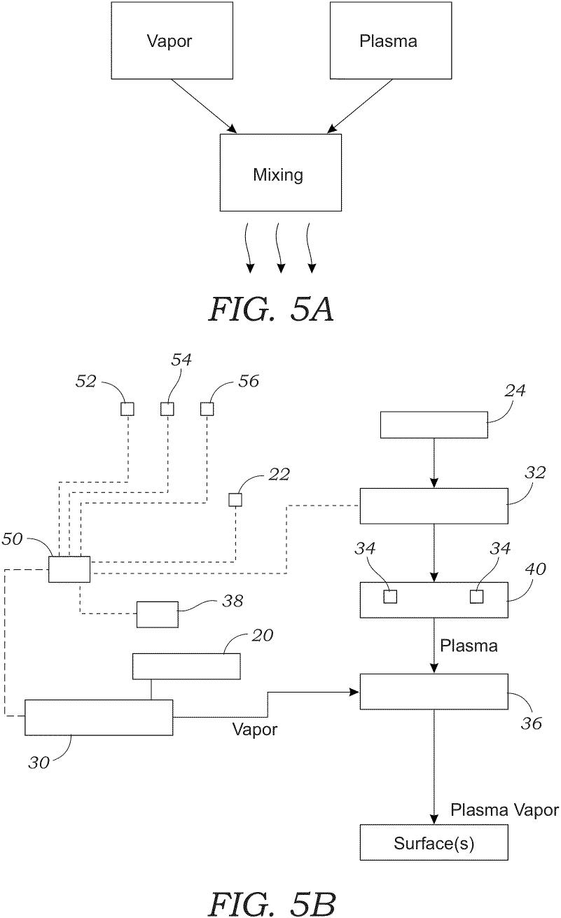

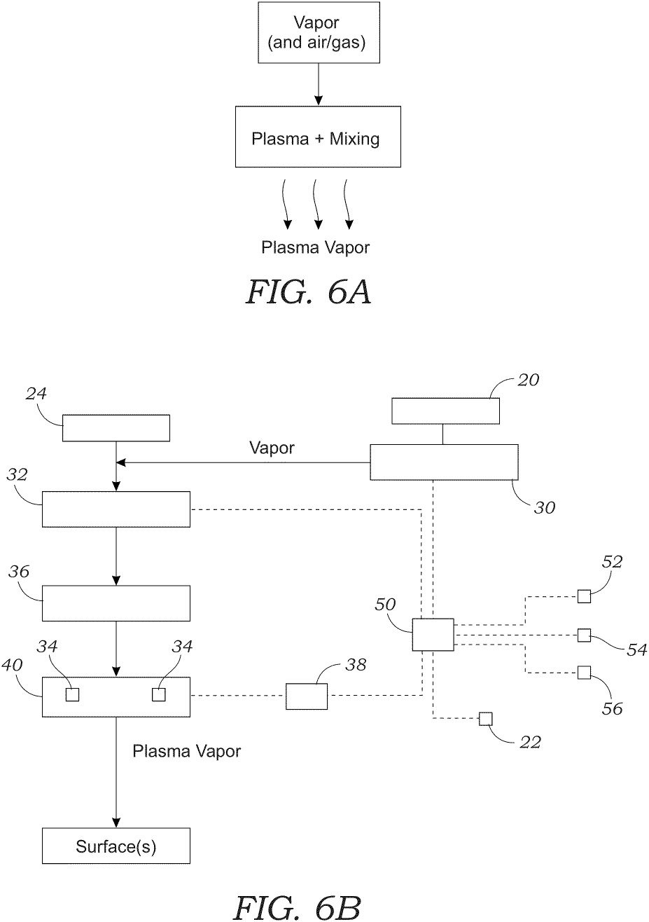

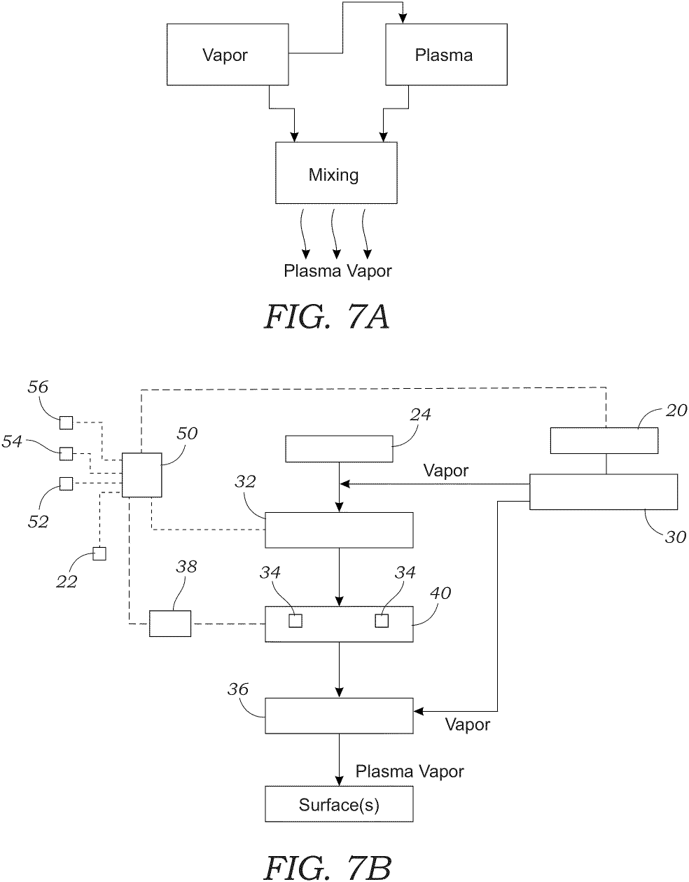

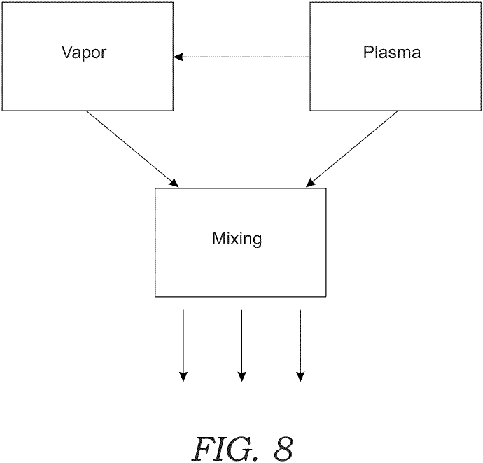

In some embodiments such as illustrated in FIG. 1B the plasma vapor dispensing device 10 includes a mixing chamber 36 where the liquid (e.g., water) vapor created by the aerosolizer/vaporizer 30 is mixed with air containing reactive species generated by the plasma generating electrode(s) 34. Thus, in this particular embodiment, the plasma is generated with air which is then mixed downstream in the mixing region 36. This is schematically illustrated in FIGS. 5A and 5B. In another embodiment such as that illustrated in FIGS. 6A and 6B, however, the air may be combined with the water/liquid vapor in a mixing region 36 and the plasma generated within the mixture. In this configuration, the gas/liquid vapor mixture is exposed to the plasma generating electrode(s) 34. That is to say, the vapor is not added after plasma formation as the vapor/air mixture is exposed to the electrodes 34 in the plasma generating region 40. The mixing region 36 may include dedicated chamber or volume in which mixing takes place. The mixing region 36 may also be formed as part of a flow path or multiple flow paths within the plasma vapor dispensing device 10. In some embodiments, the mixing region 36 may be combined with the plasma generating region 40 such that plasma generation and mixing takes place in the same region of the plasma vapor dispensing device 10. FIGS. 7A and 7B illustrate yet another alternative mode of operation in which a portion of the vapor is shunted to the plasma generating region 40 to generate plasma while a remaining portion of vapor is then mixed with the formed plasma vapor to adjust the concentration of reactive species therein or the final humidity level of the plasma vapor.

FIG. 1D illustrates an alternative embodiment of the plasma vapor dispensing device 10. In this embodiment, air is pulled from the external environment via the air intake unit 32 and plasma is generated in the plasma generating region 40 by the electrodes 34. The plasma is directed at the fluid contained in the reservoir 20 (e.g., water). For example, the plasma that is formed is directed over the surface of the fluid contained in the reservoir 20. The plasma may also be directed into or through (e.g., bubbled) the fluid. This creates an “activated” fluid that contains the components of the cold plasma. An optional mixer (not shown) may be provided in the reservoir 20 to circulate and mix the activated fluid. This embodiment creates a reservoir 20 that contains activated fluid that retains the beneficial cold plasma components and can be vaporized on-demand and used. For example, the plasma can periodically be generated to create new batches of activated fluid within the reservoir 20. To create the plasma vapor, the aerosolizer/vaporizer 30 is activated and forms the plasma vapor which can be directed on the surface(s) using the outlet(s) 26 as explained in prior embodiments. One or more optional fans or air/pumps 28 may be used to assist in exhausting or outputting the plasma vapor from the plasma vapor dispensing device 10 and onto the desired surface(s).

It should be appreciated that all or some of the plasma-contacting and/or vapor-contacting surfaces within the dispensing device 10 can be made of materials or surfaces that facilitate the generation or maintenance of plasma activated species within the plasma and/or vapor. Such materials or surfaces may include silicon nitride and the like.

FIGS. 5A, 6A, and 7A schematically illustrate three different manners of producing plasma vapor using the plasma vapor dispensing device 10. FIG. 5A illustrates an embodiment where gaseous plasma (e.g., in the air or in an air/gas mixture) is formed and combined with vapor from the liquid (e.g., water) entering a mixing region 36 to provide plasma vapor. FIG. 5B illustrates schematically the setup of the plasma vapor dispensing device 10. As seen in FIG. 5B, the air intake unit 32 sends air to the plasma generating region 40 where the electrodes 34 create the plasma. Separately, the aerosolizer/vaporizer 30 creates the vapor which is combined with the now created plasma in a mixing region 36 which creates the plasma vapor that can be exhausted or output onto the surface(s). FIG. 6A illustrates another mode of operation in which the vapor infused air/gas created from the aerosolizer/vaporizer 30 enters the plasma generating region 40 that contains the plasma generating electrode(s) 34 and is then exhausted or outputted/ejected directly as plasma vapor. FIG. 6B illustrates schematically the setup of the plasma vapor dispensing device 10. As seen in FIG. 6B, the vapor is generated by the aerosolizer/vaporizer 30 enters an optional mixing region 36 that mixes with vapor with incoming air from the air intake unit 32. Note that the mixing region 36 may be omitted in some embodiments and the ambient air and generated vapor being mixed using the air intake unit 32. The vapor infused air/gas then enters the plasma generating region 40 where the electrodes 34 create the plasma vapor which is then exhausted or output on the surface(s). FIGS. 7A and 7B illustrates a combination of the delivery schemes of FIGS. 5A and 6A where a fraction of the liquid vapor is shunted into the plasma generating region 40, while the rest of the liquid vapor is shunted to the mixing region 36 where the liquid vapor mixes with the plasma vapor to produce a plasma vapor of the desired concentration. In addition, the setup of FIGS. 7A and 7B allow not only for the tuning or adjustment of the plasma vapor reactive species, it also enables the tunability of the humidity level of the final plasma vapor. Different humidity levels may be desired for different applications or uses. These approaches provide a variety of options for plasma vapor composition and can also be used to ensure a long life for the plasma generation stage.

As seen in FIGS. 1B, 1C, 1D, 5B, 6B, 7B, the plasma vapor dispensing device 10 includes a controller 50 that includes one or more microprocessors or other control circuitry that is used to operate the various components. The controller 50, for example, may operate or control the aerosolizer/vaporizer 30, the plasma generating electrode(s) 34 via the high voltage power source(s) 38, and the air intake unit 32. The controller 50 may also receive signals or data from sensors 52, 54, 56 as explained herein (and level sensors 22). The plasma vapor dispensing device 10 may optionally include one or more sensors 52 which are used to automatically trigger the activation of the plasma vapor dispensing device 10. These may be proximity or infrared sensors 52 which sense the closeness of object near the plasma vapor dispensing device 10. For example, putting one’s hands close to the plasma vapor dispensing device 10 would trigger the generation of water vapor by the aerosolizer/vaporizer 30, activate the air intake unit 32, and the plasma generating electrode(s) 34. The sensor(s) 52 interface with the controller 50 which can be used to run a pre-determined sequence of operations. The sequence may be timed based on the first triggering by the sensor(s) 52. Alternatively, operations may stop after the person removes his/her hands or other objects which is detected by the sensor(s) 52. Of course, as a substitute for the proximity sensor 52, a simple button or activation switch may be provided to initiate the activation of the plasma vapor dispensing device 10.

The plasma vapor dispensing device 10 may also include optional plasma sensors 54 (FIGS. 1B, 1C, 1D, 5B, 6B, 7B) for monitoring plasma quality. These may include, by way of example, optical emission spectroscopy (OES) or other brightness/optical sensors. The plasma vapor dispensing device 10 may also include surface analysis sensors 56 that analyze the surface(s) to be treated. These may include ultraviolet or “black lights” that look for an indication of oil or virus content on surfaces. For example, live viruses and virus RNA can be visualized via feedback such as fluorescent genetic tags or similar techniques.

To use the plasma vapor dispensing device 10 for disinfecting hands (or an object), a person places his or her hands (or an object to be sterilized) near the outlet(s) 26 of the plasma vapor dispensing device 10. The proximity sensor(s) 52 sense the presence of the hands or object and initiates the generation of cold plasma vapor for sanitizing purposes. As explained herein, air from the surrounding environment is drawn into the plasma vapor dispensing device 10 and water vapor is also generated using the aerosolizer/vaporizer 30. The air then exposed to one or more plasma generating electrode(s) 34. This exposure may occur prior to mixing with generated water vapor or the exposure may occur after the generated water vapor has been mixed with the air. The now-formed plasma vapor is then outputted/ejected out of the one or more outlets 26 of the plasma vapor dispensing device 10 and onto the hands of the user (or other obj ect/surface).

In the embodiment of FIG. 1D, to use the plasma vapor dispensing device 10 for disinfecting hands (or an object), a person places his or her hands (or an object to be sterilized) near the outlet(s) 26 of the plasma vapor dispensing device 10. The proximity sensor(s) 52 sense the presence of the hands or object and initiates the generation of cold plasma vapor for sanitizing purposes. Here, since the liquid contained in the reservoir is activated with plasma, the aerosolizer/vaporizer 30 generates the plasma vapor that is then exhausted or output from the one or more outlets and on the hands of the user (or other object/surface).

Notably, the plasma vapor that is generated onto the hands or other skin surface is comfortable and not hot. The plasma vapor may have a temperature that is around ambient temperatures. If an object is the target surface of the plasma vapor dispensing device 10, the object may include medical devices, instruments, utensils, or any surface that requires sanitation for any application (including medicine, food, agriculture, public/private spaces, personal protective equipment (PPE)), and the like.

The plasma vapor may also be collected in a vessel for immediate or later use. This vessel may be made from any material including glass, metal, polymers or plastics, and the like as well as having surface(s) that would help maintain the presence of the plasma-activated media. The plasma vapor will likely condense within the vessel as a liquid. The vessel may then be closed for better containment and may also be filled or positively pressured with non-reactive gas or gas that helps maintain the advantageous contents of the plasma-activated liquid.

While embodiments of the present invention have been shown and described, various modifications may be made without departing from the scope of the present invention. The number, type, and shapes of electrodes 34 may vary. For example, additional and/or larger electrodes 34 may be used to generate larger volumes of plasma vapor or plasma vapor with increased concentration of RON and ROS. In addition, for the embodiment of FIG. 1D which draws ambient air in to create the plasma which activates the fluid 21 in the reservoir 20, it is possible to change the air/gas source to a tank 23 like that of FIG. 1C which is used to release air/gas(es) for the generation of plasma and activate the fluid 21. The invention, therefore, should not be limited, except to the following claims, and their equivalents.

What is claimed is:

1. A plasma vapor dispensing device comprising:
a fluid reservoir configured hold liquid fluid therein;
an aerosolizer/vaporizer configured to generate a vapor of the liquid contained in the fluid reservoir;
an air intake unit that pumps or transports ambient air into the plasma vapor dispensing device from the surrounding environment;
one or more plasma generating electrodes operatively coupled to a high voltage power source and disposed in the plasma device at a plasma generating region to generate plasma in the presence of ambient air, the liquid vapor, or a mixture thereof to generate a plasma vapor; and
one or more outlets that exhaust or output the plasma vapor from the plasma vapor dispensing device.

2. The plasma vapor dispensing device of claim 1, wherein the liquid comprises water.

3. The plasma vapor dispensing device of claim 1, wherein the aerosolizer/vaporizer comprises a vibrating plate or substrate.

4. The plasma vapor dispensing device of claim 1, further comprising one or more proximity sensors, plasma sensors, and/or surface analysis sensors.

5. The plasma vapor dispensing device of claim 1, further comprising a controller configured to control one or more of the aerosolizer/vaporizer, air intake unit, and the one or more plasma generating electrodes.

6. The plasma vapor dispensing device of claim 1, wherein the one or more plasma electrodes comprises a single electrode or a plurality of single electrodes or electrode pairs.

7. The plasma vapor dispensing device of claim 1, wherein the frequency and/or peak-to-peak voltage applied to the one or more plasma generating electrodes is adjustable.

8. A plasma vapor dispensing device comprising:
a fluid reservoir configured hold liquid fluid therein;
a source of gas;
an aerosolizer/vaporizer configured to generate a vapor of the liquid contained in the fluid reservoir from gas from the source of gas traveling through or over the liquid fluid;
one or more plasma generating electrodes operatively coupled to a high voltage power source and disposed in the plasma device at a plasma generating region to generate plasma in the gas, the liquid vapor, or a mixture thereof to generate a plasma vapor; and
one or more outlets that exhaust or output the plasma vapor from the plasma vapor dispensing device.

9. The plasma vapor dispensing device of claim 8, wherein the source of gas is pressurized.

10. (canceled)

11. A plasma vapor dispensing device comprising:
a fluid reservoir configured hold liquid fluid therein;
an aerosolizer/vaporizer configured to generate a vapor of the liquid contained in the fluid reservoir;
an air intake unit that pumps or transports ambient air into the plasma vapor dispensing device from the surrounding environment;
one or more plasma generating electrodes operatively coupled to a high voltage power source and disposed in the plasma device at a plasma generating region to generate plasma in the presence of ambient air, the liquid vapor, or a mixture thereof to generate a plasma vapor; and
one or more outlets that exhaust or output the plasma vapor from the plasma vapor dispensing device.

12. The plasma vapor dispensing device of claim 11, wherein the one or more plasma generating electrodes generate plasma at the plasma generating region in the presence of ambient air and plasma is directed onto or into the fluid contained in the fluid reservoir.

13. The plasma vapor dispensing device of claim 11, further comprising one or more proximity sensors, plasma sensors, and/or surface analysis sensors.

14. The plasma vapor dispensing device of claim 11, further comprising a controller configured to control one or more of the aerosolizer/vaporizer, air intake unit, and the one or more plasma generating electrodes.

15. The plasma vapor dispensing device of claim 11, wherein the frequency and/or peak-to-peak voltage applied to the one or more plasma generating electrodes is adjustable.

16. The plasma vapor dispensing device of claim 1, wherein one or more surfaces of the plasma vapor dispensing device that contact the vapor and/or plasma are made of materials that facilitate the generation or maintenance of plasma activated species within the vapor and/or plasma.

17. A method of using plasma vapor to disinfect or sterilize a surface using a plasma vapor dispensing device comprising:
generating liquid vapor using_{:} (1) a vibrating substrate or surface in the plasma vapor dispensing device, or (2) a source of gas that is delivered into or over liquid contained in the plasma vapor dispensing device;
generating a plasma vapor in the presence of one or more of the ambient air, the source of gas, the liquid vapor, or a mixture thereof with one or more plasma generating electrodes operatively coupled to a high voltage power source and disposed in the plasma device; and
exhausting or outputting the plasma vapor from the plasma vapor dispensing device via one or more outlets onto the surface.

18. The method of claim 17, wherein the surface comprises skin of a biological organism or the surface of an object or vessel.

19. (canceled)

20. The method of claim 17, further comprising sensing the presence of the surface near the plasma vapor dispensing device using one or more proximity sensors, plasma sensors, and/or surface analysis sensors.

21. (canceled)

22. The method of claim 17, wherein plasma vapor is generated in the presence of ambient air and subsequently mixed with the liquid vapor.

23. The method of claim 1724, wherein the plasma vapor is generated by passing a portion of the liquid vapor by the one or more plasma electrodes.

24. The method of claim 23, further comprising combining a remaining portion of the liquid vapor with the plasma vapor prior to exhausting or outputting the plasma vapor.

25. (canceled)
